(19) United States
(12) Patent Application Publication
(10) Pub. No.: US 2015/0374896 A1
(43) Pub. Date: Dec. 31, 2015

(54) Needle dislodgement and blood leakage detection device

(71) Applicant: Yi-Chun Du, Tainan City (TW)

(72) Inventors: Yi-Chun Du, Tainan City (TW); Bee Yen Lim, Klang (MY); Ming-Jui Wu, Kaohsiung City (TW); Chia-Hung Lin, Kaohsiung City (TW)

(21) Appl. No.: 14/558,688

(22) Filed: Dec. 2, 2014

(30) Foreign Application Priority Data
Jun. 25, 2014 (TW) 103121918

(51) Int. Cl. A61M 1/36; A61M 5/50

(52) U.S. Cl. CPC A61M 1/3656; A61M 5/5086; A61M 2205/15

(57) ABSTRACT

A needle dislodgement and blood leakage detection device includes a sensor assembly including a flexible sensor and an alarm device to which the sensor assembly is mounted. The flexible sensor includes two blood leakage detection electrodes that circumferentially surround a needle piercing site. The blood leakage detection electrodes are contactable by a needle in a manner of crossing therebetween to get shorted or are contactable with blood leaking out of the needle piercing site to get shorted. The alarm device is positionable on a body surface of a patient and is electrically connected to the blood leakage detection electrodes so as to issue an alarm message when the detection electrodes are shorted. The flexible sensor has a thin plate configuration that is flexible allows the flexible sensor to be flat attached to the patient body surface and deformable with the bending of a limb of the patient.

### BACKGROUND OF THE INVENTION

(a) Technical Field of the Invention

The present invention generally relates to a blood leakage detection device, and more particularly to a needle dislodgement and blood leakage detection device that detects needle dislodgement and blood leakage at a needle piercing site.

(b) Description of the Prior Art

When a patient receives hemodialysis, the blood must be conducted to a hemodialysis machine for filtration and treatment and thus routed back into the human body. A needle must be used to pierce into a blood vessel to drain the blood and once the hemodialysis is completed, the needle must be removed. Since the needle used has a large diameter, the opening formed in the blood vessel during needle piercing is relatively large. Thus, after piercing and removal of a needle, dislodgement of the needle and blood leakage may often occur. If this is not timely discovered and learned by the patient or medical personnel, mass bleeding of the patient may occur. In view of such problems, products that detect blood leakage have been developed and proposed, such as the blood leakage detection device disclosed in US Patent Application No. 2008/0249487.

Such a known blood leakage detection device identifies blood leakage by detecting if there is blood attached to a tip of an optic fiber probe. If blood is attached to locations other than the tip of the optic fiber probe, it will be determined that there is no blood leakage. Thus, to correctly detect if blood leakage occurs at a needle piercing site in using the device, it is necessary to align the tip of the optic fiber probe with the needle piercing site. This is a single-spot detection process and the tip of the optic fiber probe may be easily shifted away due to a single wire of the optic fiber probe being pulled and stretched thereby leading to false detection and causing undesired delay of issuing blood leakage alarm. Further, the optic fiber probe must be arranged on the same parallel line as the needle and may thus press on the area where the needle pierces into a fistula, making it easily compress the fistula and the needle. Further, the known blood leakage detection device can only detects the leakage of blood and cannot be used to detect needle dislodgement where the needle undeservedly slides off. It also cannot issue an alarm message when the known detection device gets detached.

### SUMMARY OF THE INVENTION

Thus, an object of the present invention is to provide a needle dislodgement and blood leakage detection device that does not compress a fistula and accurately detects blood leakage and needle dislodgement.

Another object of the present invention is to provide a needle dislodgement and blood leakage detection device that gives off an alarm when gets detached.

Therefor, the present invention provides a needle dislodgement and blood leakage detection device, which is applicable to detection of blood leakage at a needle piercing site on a body surface of a patient and comprises an alarm device and a sensor assembly that is detachably and electrically connected to the alarm device. The sensor assembly comprises a flexible sensor that is positionable on the patient body surface and is flexibly deformable. The flexible sensor comprises a flexible substrate and two blood leakage detection electrodes mounted on a surface of the flexible substrate. The flexible substrate comprises an alignment portion that circumferentially delimits and defines a needle piercing space that covers the needle piercing site. The blood leakage detection electrodes respectively comprise first detection sections that are mounted to a top surface of the alignment portion and spaced from each other. The first detection sections are contactable by a needle in a manner of crossing therebetween and thus shorted or are contactable with blood leaking out of the needle piercing site to get conducted with each other and thus shorted. The alarm device comprises a housing unit that is detachably attachable to the patient body surface, a conductive terminal that is mounted to the housing unit to receive the sensor assembly to detachably connect thereto for being in signal connection with the blood leakage detection electrodes, a control unit that is mounted in the housing unit and electrically connected to the conductive terminal, and an alarm unit that is drivable by the control unit to generate an alarm message. The control unit is operable to drive the alarm unit to issue the alarm message when the first detection sections are conducted with each other and thus shorted.

The efficacy of the present invention is that with the flexible thin plate like flexible sensor arrangement of the sensor assembly, the flexible sensor is allowed to deform in compliance with the bending of limb so as to maintain laid flat on the body surface of the patient without causing compression of fistula and the needle and is applicable to detection of blood leakage in a blood transfusion process and a bleeding stopping process, and is thus very practical and useful.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view showing a needle dislodgement and blood leakage detection device according to a first embodiment of the present invention.

FIG. 2 is a bottom view of a flexible sensor of the first embodiment of the present invention.

FIG. 3 is a cross-sectional view of the flexible sensor of the first embodiment of the present invention.

FIG. 4 is a functional block diagram of the first embodiment of the present invention.

FIG. 5 is a schematic view illustrating installation and use of the first embodiment of the present invention on the body of a patient.

FIG. 6 is a top plan view of a flexible sensor of a needle dislodgement and blood leakage detection device according to a second embodiment of the present invention.

FIG. 7 is a bottom view of the flexible sensor of the second embodiment of the present invention.

FIG. 8 is a top plan view of the flexible sensor of the second embodiment of the present invention, illustrating a different form of the flexible sensor.

FIG. 9 is an exploded view showing a needle dislodgement and blood leakage detection device according to a third embodiment of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

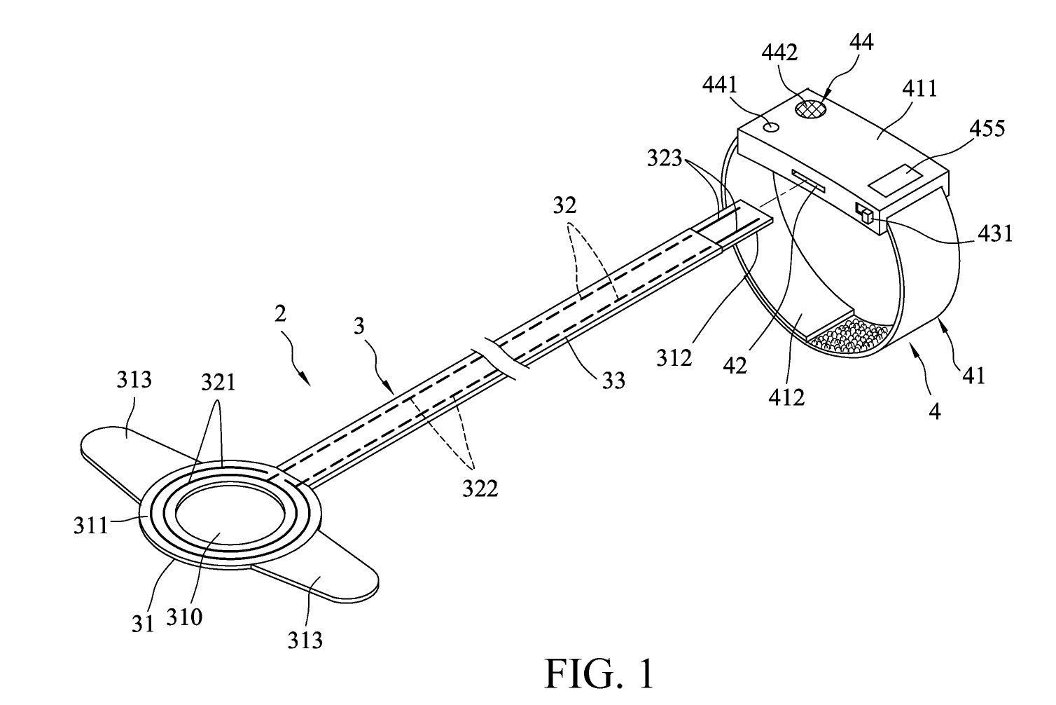

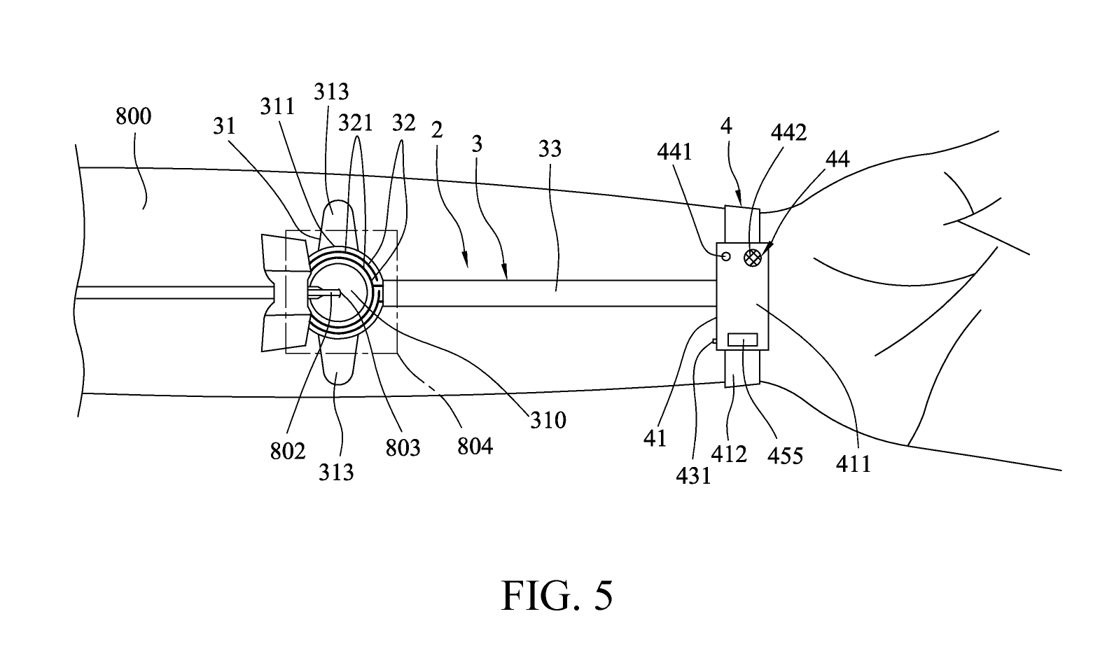

As shown in FIGS. 1 and 5, a needle dislodgement and blood leakage detection device according to a first embodiment of the present invention is applicable to installation on an arm of a patient 800 to detect dislodgement of a needle 802 piercing into the patient and to detect and issue an alarm of blood leakage at a needle piercing site 803 where the needle 802 pierces. In the instant embodiment, the patient may be, but not limited to, a hemodialysis patient that needs hemodialysis and/or blood transfusion.

The needle dislodgement and blood leakage detection device comprises a sensor assembly 2 and an alarm device 4 that is detachably and electrically connected to the sensor assembly 2.

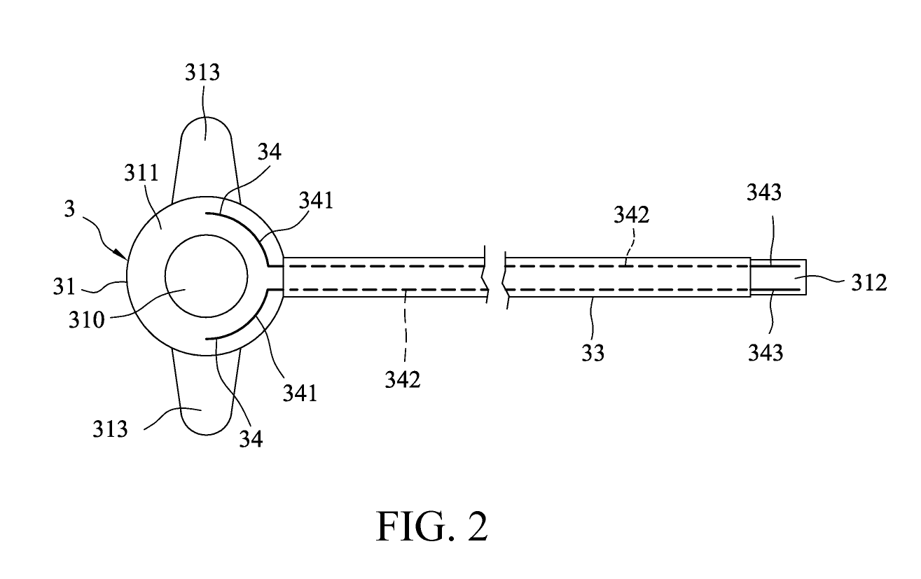

As shown in FIGS. 1, 2, and 3, the sensor assembly 2 comprises a flexible sensor 3 that is attachable to a body surface of the patient 800 and is detachably and electrically connected to the alarm device 4. The flexible sensor 3 can be a thin plate like printed circuit board that is flexible and deformable and comprises a flexible substrate 31 that is flexibly deformable, two strip-like blood leakage detection electrodes 32 that are fixed, in a spaced and covered manner, to a top surface of the flexible substrate 31, two strip-like body surface detection electrodes 34 that are fixed, in a spaced and covered manner, to a bottom surface of the flexible substrate 31, and an insulation film 33 that encloses and is fixed to the outside of the flexible substrate 31 and covers the blood leakage detection electrodes 32 and the body surface detection electrodes 34.

In the instant embodiment, the flexible substrate 31 is made of polyester (PET) and may also be made of polyimide (PI), but not limited to these materials. The flexible substrate 31 comprises an annular plate like alignment portion 311 that circumferentially delimits and defines a needle piercing space 310 that covers the needle piercing site 803, an elongate plate like extension portion 312 that integrally and outwards extends from an outer circumference of the alignment portion 311, and two flexible wing portions 313 that are arranged to extend, in a spaced and symmetric manner, from the alignment portion 311 to be located on opposite sides of the extension portion 312.

The blood leakage detection electrodes 32 are each composed of a first detection section 321 extending along a circumference of the alignment portion 311, a first extension section 322 extending in a lengthwise direction of the extension portion 312, and a first conduction section 323 formed on an end section of the extension portion 312. The first detection sections 321 may get shorted due to conduction resulting from contact with blood or engagement with a needle 802.

The body surface detection electrodes 34 are caused to electrically engage the body surface of the patient 800 when the flexible substrate 31 is set in engagement with the body surface of the patient 800. The body surface detection electrodes 34 are each composed of a second detection section 341 extending in a direction of extension of the alignment portion 311, a second extension section 342 extending in the lengthwise direction of the extension portion 312, and a second conduction section 343 formed on an end section of the extension portion 412. The second detection sections 341 are individually and electrically engageable with the body surface to get shorted as being conducted by a body tissue so as to form, together with the alarm device 4, a detection circuit.

In the instant embodiment, the blood leakage detection electrodes 32 and the body surface detection electrodes 34 are respectively fixed, in a covered manner, to the top and bottom surfaces of the flexible substrate 31 by means of printing so as to each show a form of a thin film. However, they can be embodied by means of film coating techniques to form on the flexible substrate 31; however, the manufacture is not limited to these processes. It is noted here that the lay-out of the blood leakage detection electrodes 32 and the lay-out of the body surface detection electrodes 34, which are shown in the drawings associated with the instant embodiment, can be modified according to the requirements of applications, such as adjusting the line width, spacing, and extension distribution, and it is possible to lay, through a simple increase of number, a large number of blood leakage detection electrodes 32 and body surface detection electrodes 34 on the flexible substrate 31. Thus, in a practical embodiment, the shapes and numbers of the blood leakage detection electrodes 32 and the body surface detection electrodes 34 are not limited to what illustrated in the drawings.

The insulation film 33 is covered and fixed to the outside of the extension portion 312 to enclose and shield the first extension sections 322 and the second extension sections 342 with only the first detection sections 321 and the first conduction sections 323 of the blood leakage detection electrodes 32 exposed outside and also the second detection sections 341 and the second conduction sections 343 of the body surface detection electrodes 34 exposed outside.

As shown in FIGS. 1, 3, and 4, the alarm device 4 comprises a housing unit 41 and a conductive terminal 42, a power supply unit 43, an alarm unit 44, a control unit 45, and a communication unit 46 mounted to the housing unit 41. The housing unit 41 comprises a housing body 411 and a looping member 412 that is mounted to the housing body 411 for looping around a limb of the patient 800 (as illustrated in FIG. 5). The conductive terminal 42, the power supply unit 43, the alarm unit 44, the control unit 45, and the communication unit 46 are arranged inside the housing body 411.

In the instant embodiment, the looping member 412 is composed of two fastening straps that are mounted to the housing body 411 and are fastenable to each other through for example hook-and-loop engagement. However, a variety of different looping members for looping around a limb of a patient 800 are available and these are not a novel part of the present invention so that the embodiment of the present invention is not limited to the fastening means described above.

The conductive terminal 42 is mounted to expose on a surface of the housing body 411 to allow the end section of the extension portion 312 of the flexible substrate 31 to be inserted, in a detachable manner, therein and coupled thereto thereby electrically engageable with the first conduction sections 323 and the second conduction sections 343. The power supply unit 43 supplies electrical power necessary for the operations of the alarm unit 44, the control unit 45, and the communication unit 46 and comprises a power supply switch 431 that is mounted to expose outside the surface of the housing body 41.

The alarm unit 44 is drivable by the control unit 45 to issue alarm messages indicating blood leakage and dislodgement of the needle 802, and also gives off an alarm message indicating detachment of the body surface detection electrodes 34. In the instant embodiment, the alarm unit 44 comprises an alarming element 442 and a light-emitting element 441 exposed outside a surface of the housing body 411. The alarming element 442 is drivable to give off an alarm message of loud sound and the light-emitting element 441 is drivable to give off an alarm message of bright lighting.

The communication unit 46 is communicateable with an electronic device 900 for transmission of data. In the instant embodiment, the communication unit 46 is operable to communicate with the electronic device 900 through wireless communication, such as communication techniques including Bluetooth, ZigBee, NFC, and RDS. In an embodiment, cabled communication measures may be used for communication with the electronic device 900. The electronic device 900 can be an electronic device carried by medical personnel or a family member of the patient 800, such as a mobile phone, a tablet computer, and a notebook computer, or may be a computer or other alarm facility installed in a medical station, but not limited to these devices.

The control unit 45 comprises an electrical signal detection module 451, a power interruption protection module 452, a shorting detection module 453, a detachment detection module 456, and a control module 454. The electrical signal detection module 451 is in signal connection with the conductive terminal 42 and may transmit an electrical detection signal to the conductive terminal 42 to be applied, via the conductive terminal 42, to one of the blood leakage detection electrodes 32 of the flexible sensor 3.

The power interruption protection module 452 is in signal connection with the electrical signal detection module 451 and the control module 454 and is drivable by the control module 454 to cause the electrical signal detection module 451 to stop outputting the electrical detection signal.

The shorting detection module 453 is in electrical connection with the conductive terminal 42 and the control module 454 and is electrically connected, via the conductive terminal 42, to the blood leakage detection electrodes 32 so as to detect, at the time when the blood leakage detection electrodes 32 are shorted, and output a shorting signal to the control module 454.

The detachment detection module 456 is in signal connection, via the conductive terminal 42, with the body surface detection electrodes 34 so as to form, in combination with the body surface detection electrodes 34 and the body surface, a detection circuit at a condition where the body surface detection electrodes 34 are attached to the body surface and the power supply unit 43 is activated on and to generate a detachment signal at the time when the detection circuit is being opened due to one of the body surface detection electrodes 34 being caused to separate from the body surface resulting from detachment of the flexible substrate 31 from the body surface.

The control module 454 is in signal connection with the alarm unit 44 and the communication unit 46 and is triggerable by the shorting signal to drive and enable the power interruption protection module 452, making the power interruption protection module 452 cause the electrical signal detection module 451 to stop output the electrical detection signal and driving the alarm unit 44 to issue an alarm message indicating blood leakage or needle dislodgement, meaning driving the light-emitting element 441 to give off alarm light, and driving the alarming element 442 to give off an alarm sound in order to notify the patient 800 and other people nearby. The control module 454 is also triggerable by the detachment signal to drive the alarm unit 44 to issue an alarm message indicating detachment of the flexible sensor 3 in order to notify the patient 800 and other people nearby. Further, the control module 454, when triggered by the blood leakage signal and the detachment signal, drives the communication unit 46 to transmit an alarm signal to the electronic device 900 to drive the electronic device 900 to issue an alarm message to notify related people.

The control module 454 comprises a reset button 455 mounted to expose on the surface of the housing body 411, so that the reset button 455, when actuated, disables the power interruption protection module 452 so as to allow the electrical signal detection module 451 to generate and output an electrical detection signal to the conductive terminal 42 again.

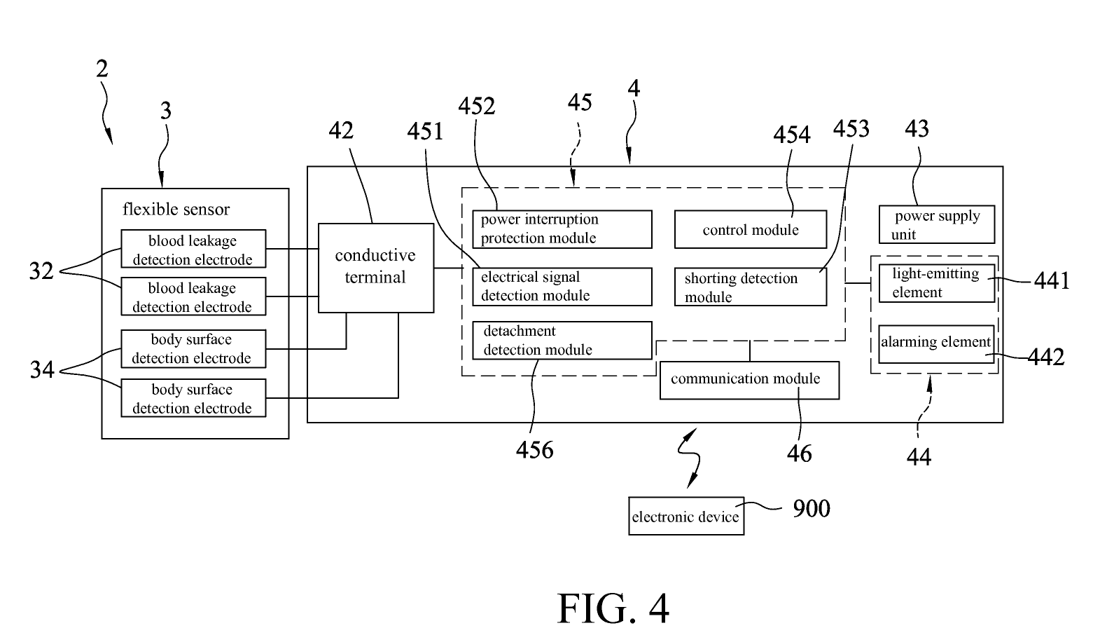

As shown in FIGS. 1, 3, and 5, in the use of the needle dislodgement and blood leakage detection device of the present invention, the flexible sensor 3 of the sensor assembly 2 is positioned and laid flat on the body surface of the patient 800, such as using an adhesive tape to fix the wing portions 313 to the body surface, with the portion of the patient 800 where a needle is to pierce for blood transfusion being exposed in the range of the needle piercing space 310 of the alignment portion 311 and the second detection sections 341 of the body surface detection electrodes 34 attached to the body surface of the patient 800. Under such a condition, medical personnel may perform piercing with a needle 802 in the needle piercing space 310 with an insulation portion of the needle 802 positioned on and fixed to a top surface of the alignment portion 311 and a piece of gauze 804 be positioned and retained on the needle piercing site 803 and the detection sections 321. Afterwards, the flexible sensor 3 is inserted into and electrically connected with the conductive terminal 42 of the alarm device 4. Finally, the looping member 412 is set to loop around and positioned on the limb into which the needle pierces and as such, the installation of the entire device is completed. When the power supply switch 431 is switched on, the control unit 45 (shown in FIG. 4) is enabled and starts to detect if the blood leakage detection electrodes 32 are shorted and if circuit opening is identified between the body surface detection electrodes 34.

Generally, the needle 802 has a length of around 2.5 cm and the length of the portion exposed outside the body of the patient 800 is usually less than 1 cm. Further, before blood leakage occurs at the needle piercing site 803, needle dislodgement that the needle 802 slides off the body of the patient 800 often occurs first, whereby when the extent that the needle 802 slides off is excessively large, a large opening is formed in the needle piercing site 803 to allow for the occurrence of blood leakage at the needle piercing site 803.

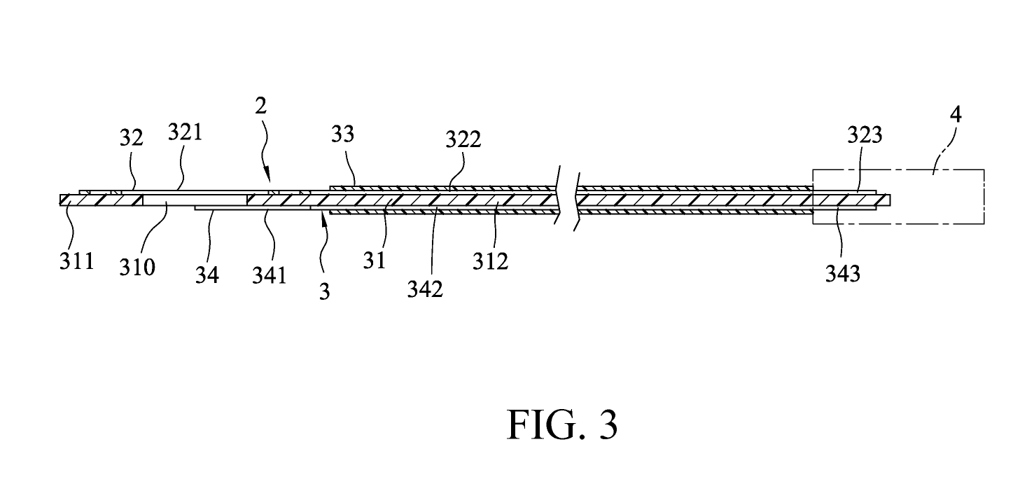

As shown in FIGS. 3, 4, and 5, in case that the needle 802 is not properly fixed or the adhesive tape for fixing gets loosened due to pulling and stretching of the limb of the patient 800, the needle 802 may start to slide off the body and move to the location above the first detection sections 321 so as to contact and make the first detection sections 321 conducted with each other, such as the needle 802 sliding off by a length greater than 1 cm, whereby the control unit 45 is triggered and immediately drives the power interruption protection module 452 to stop the electrical signal detection module 451 for further outputting the electrical detection signal and to drive the alarm unit 44 to issue an alarm message and also drive the communication unit 46 to transmit the alarm message to the electronic device 900 so as to notify the patient 800, the family member, or medical personnel to take necessary step for handling so that it is possible to issue an alarm before the actual dislodgement of the needle 802 and bleeding take place.

In case that blood leakage occurs, the gauze 804 absorbs the blood. Since the gauze 804 is stacked on the first detection sections 321 of the blood leakage detection electrodes 32, the blood leakage detection electrodes 32 are conducted with each other and thus shorted by the blood absorbed in the gauze 804 and the control module 454 is immediately triggered to drive the power interruption protection module 452 to stop the electrical signal detection module 451 for outputting the electrical detection signal and to drive the alarm unit 44 to issue an alarm message and also to transmits, via the communication unit 46, the alarm signal to the electronic device 900 so as to notify the patient 800, the family member, or medical personnel to take necessary step for handling to prevent the patient 800 from losing too much blood.

With the above-described arrangement of two-stage detection of blood leakage by detecting the needle 802 sliding off and detecting blood leakage at the needle piercing site 803, it is possible to provide an advance alarm at the stage of needle dislodgement that occurs before actual blood leakage takes place and it is also possible to provide an alarm again when blood leakage actually starts. With such an arrangement of double detection and alarming, it is possible to reduce clinic risk and ensure the security of the patient 800.

In the above embodiment, illustration of the present invention is made for an example that the needle dislodgement and blood leakage detection device is used to detect if blood leaks during the process of piercing a needle 802 for blood transfusion. However, in an embodiment, the needle dislodgement and blood leakage detection device of the present invention may be used to detect blood leakage occurring in a bleeding stop process after the needle 802 is removed.

When the needle dislodgement and blood leakage detection device detects blood leakage, the flexible sensor 3 that is attached with the blood is pulled off the alarm device 4 and a fresh flexible sensor 3 as a substitute is installed. The reset button 455 is operated to disable the power interruption protection module 452 and to drive the electrical signal detection module 451 to re-generate and output an electrical detection signal for being used again for detection of blood leakage.

Further, when the flexible substrate 31 is separated from the body surface of the patient 800, such as the adhesive tape for fixing getting loosened and detached, so that one of the body surface detection electrodes 34 separates from the body surface, the control unit 45 is triggered to drive the alarm unit 44 to issue an alarm message indicating the flexible sensor 3 is detached and also to drive the communication unit 46 to transmit the alarm signal to the electronic device 900 to notify related people.

With the arrangement that the flexible sensor 3 of the needle dislodgement and blood leakage detection device has a form of a flexible thin plate, the flexible sensor 3 can be positioned, in a flat laid manner, to be extremely compliant with the body surface of the patient 800 and deflecting with the bending of the limb of the patient 800 without causing any compression to fistula of blood vessel and not affecting the piercing of the needle 802, and is applicable to the detection of blood leakage during the process of the needle 802 piercing for blood transfusion and the detection of blood leakage during a process of stopping bleeding after the needle 802 is removed and the detection of needle dislodgement of the needle 802 and automatic issuance of an alarm message at the time when the flexible sensor 3 gets detached, making it very practical and useful.

In the instant embodiment, notification can be made through the communication unit 46 to persons at remote sites. However, in an embodiment, it is not necessary to include the communication unit 46 and notification and alarming are made only through sounding and/or lighting by the alarm unit 44.

Further, in the instant embodiment, the alignment portion 311 of the flexible substrate 31 is made in the form of a circular ring; however, in an embodiment, the alignment portion 311 can be made in the form of a quadrilateral ring, a triangular ring, or rings of other geometric shapes.

In the instant embodiment, the purpose of mounting the body surface detection electrodes 34 to the bottom surface of the flexible substrate 31 is to detect if the flexible substrate 31 gets detached. However, in an embodiment, it is not necessary to include the body surface detection electrodes 34; in other words, the needle dislodgement and blood leakage detection device can only include the functions of detecting blood leakage and needle dislodgement. Under such a condition, it is not necessary for the insulation film 33 to enclose the bottom surface of the flexible substrate 31 and it can be attached to and covers the top surface of the flexible substrate 31.

It is noted here that in the instant embodiment, the body surface detection electrodes 34 are made of an electrically conductive metal and are set to be in direct electrical engagement with the body surface of the patient 800, so that the electrical conductivity of the body surface and the alarm device 4 are used in combination to form the detection circuit. Thus, when one of the body surface detection electrodes 34 is caused by the flexible substrate 31 to separate from the body surface, making the detection circuit open, meaning the resistance between the body surface detection electrodes 34 becomes infinite, the alarm device 4 drives the alarm unit 44 to issue the alarm message. However, in an embodiment, the body surface detection electrodes 34 are replaced by capacitive electrode and each capacitive electrode of the body surface detection electrodes 34 comprises a second detection section 341 and two second conduction sections 343 that are respectively and electrically connected to two ends of the second detection section 341 and are electrically connected to the conductive terminal 42. Each of the body surface detection electrodes 34 can be used individually in combination with the alarm device 4 to form an independent detection circuit. And, the second detection section 341, when contacting the body surface, generates a capacitive coupling effect with the body surface so as to induce variation of electrical signal in the detection circuit. With such an arrangement, the alarm device 4 may detect the variation of electrical signal of the detection circuit to immediately detect the event that the flexible substrate 31 separate from the body surface of the patient 800 and drive the alarm unit 44 to issue an alarm message. Since the capacitive electrode is well known and is of a variety of types, further detail is omitted here. In an embodiment, it is possible to install only one capacitive electrode type body surface detection electrode 34 on the bottom surface of the flexible substrate 31.

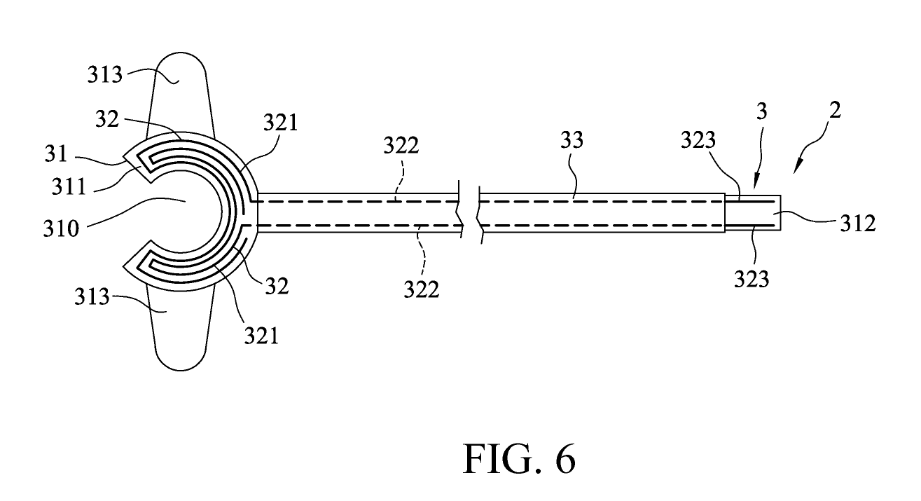

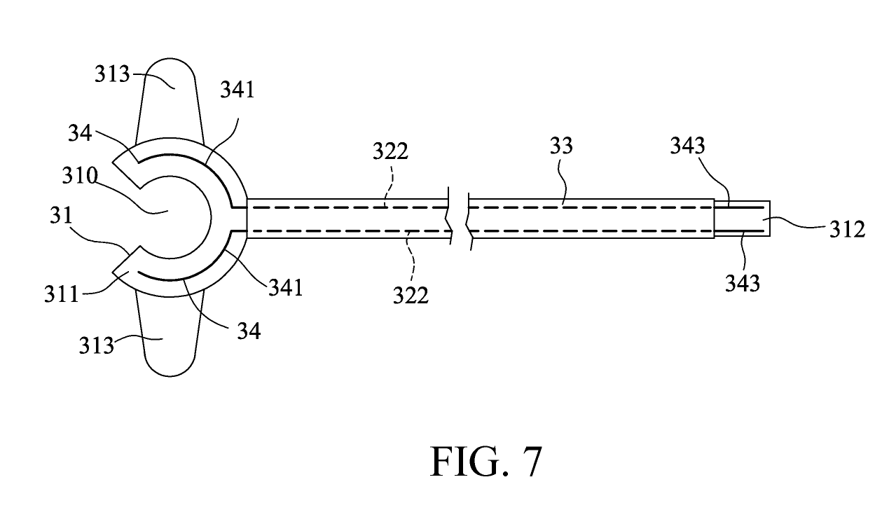

As shown in FIGS. 6 and 7, a needle dislodgement and blood leakage detection device according to a second embodiment of the present invention is different from the first embodiment in the structural design of the flexible sensor 3. For easy explanation, the following description is given to only the differences between the instant embodiment and the first embodiment.

The flexible sensor 3 similar comprises a flexible substrate 31, two blood leakage detection electrodes 32 and two body surface detection electrodes 34 respectively fixed to and covered on top and bottom surfaces of the flexible substrate 31, and an insulation film 33 enclosing and covers the outside of the flexible substrate 31. The flexible substrate 31 comprises an alignment portion 311 that is in the form of a curved elongate plate and is curved to define a needle piercing space 310 that has an opening. In the instant embodiment, the alignment portion 311 is in the form of a C-shape having an opening facing away from the extension portion 312. The blood leakage detection electrodes 32 comprise first detection sections 321 that are respectively curved and extended in an extension direction of the alignment portion 311.

The needle dislodgement and blood leakage detection device of the instant embodiment can be operated in the same way as that of the first embodiment with repeated description being omitted herein and is also applicable to the detection of needle dislodgement and blood leakage and is also applicable to the detection of blood leakage in the process of stopping bleeding after the needle 802 is removed.

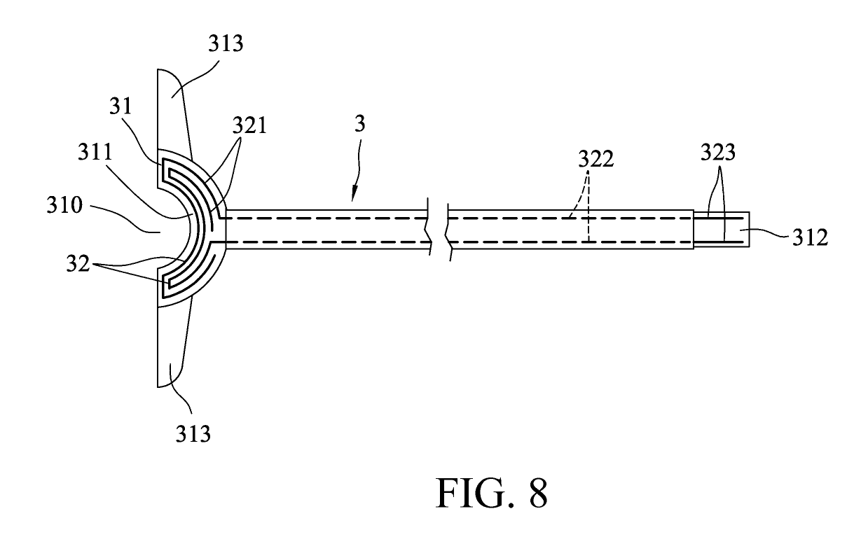

As shown in FIG. 8, another form of the flexible sensor 3 of the embodiment is illustrated, wherein the alignment portion 311 is curved to show a semicircular configuration. However, in an embodiment, the configuration of the alignment portion 311 is not limited to those mentioned above and can be in the form of a U-shape or a V-shape.

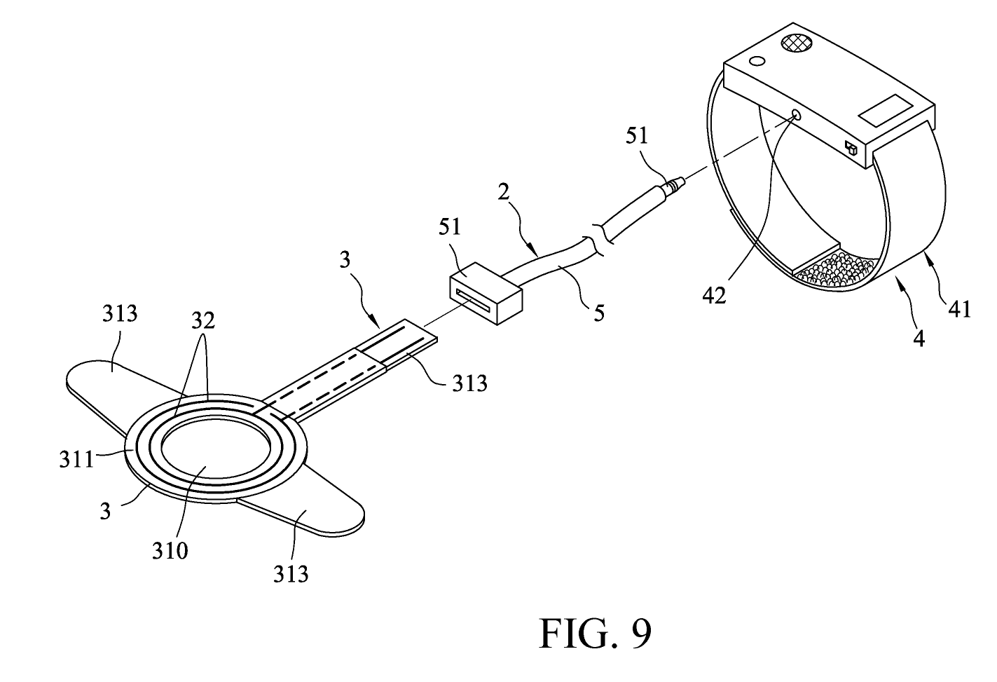

As shown in FIG. 9, a needle dislodgement and blood leakage detection device according to a third embodiment of the present invention is different from the first embodiment in the structural design of the sensor assembly 2 and the signal connection thereof with the alarm device 4.

In the instant embodiment, the sensor assembly 2 further comprises a signal cable 5 that is detachably and electrically connected to the flexible sensor 3. The signal cable 5 has two ends each provided with a connection terminal 51, of which one connection terminal 51 is provided for fitting to the flexible substrate 31 of [symbol] the flexible sensor 3 to electrically connect with the blood leakage detection electrodes 32 and the body surface detection electrodes 34 and another connection terminal 51 is provided for electrically inserting into the conductive terminal 42 of the alarm device 4 to set the blood leakage detection electrodes 32 and the body surface detection electrodes 34 in signal connection with the alarm device 4.

With the arrangement of the signal cable 5 in signal connection between the flexible sensor 3 and the alarm device 4, the installation of the needle dislodgement and blood leakage detection device is made flexible.

In summary, the flexible structural design of the flexible sensor 3 in the form of a thin plate allows the flexible sensor 3 to be compliantly and flat laid on a portion of a body surface of a patient 800 where needle piercing for blood transfusion is to be made and to be deformable with the bending of the limb of the patient 800 without causing compression on fistula and the needle and to be used in blood leakage detection during a blood transfusion process and a bleeding stopping process and to be also applicable to detection of needle dislodgement of a needle 802 and to be operable to automatically issue an alarm when getting detached. And, when the flexible sensor 3 is attached with blood or when it is removed and installed on other patients 800, it only needs to remove and install a fresh flexible sensor 3 to be operable for blood leakage detection or for use by other patients 800 without causing blood infection, making it easy to use. Thus, it is possible to reliably achieve the purpose of the present invention.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A needle dislodgement and blood leakage detection device, adapted to detect blood leakage at a needle piercing site on a body surface of a patient, comprising an alarm device and a sensor assembly that is detachably and electrically connected to the alarm device, wherein:
the sensor assembly comprises a flexible sensor that is positionable on the patient body surface and is flexibly deformable, the flexible sensor comprising a flexible substrate and two blood leakage detection electrodes mounted on a surface of the flexible substrate;
the flexible substrate comprises an alignment portion that circumferentially delimits and defines a needle piercing space that covers the needle piercing site, the blood leakage detection electrodes respectively comprising first detection sections that are mounted to a top surface of the alignment portion and spaced from each other, the first detection sections being contactable by a needle in a manner of crossing therebetween and thus shorted and being also contactable by blood leaking out of the needle piercing site to get conducted with each other and thus shorted; and
the alarm device comprises a housing unit that is detachably attachable to the patient body surface, a conductive terminal that is mounted to the housing unit to receive the sensor assembly to detachably connect thereto for being in signal connection with the blood leakage detection electrodes, a control unit that is mounted in the housing unit and electrically connected to the conductive terminal, and an alarm unit that is drivable by the control unit to generate an alarm message, the control unit being operable to drive the alarm unit to issue the alarm message when the first detection sections are conducted with each other and thus shorted.

2. The needle dislodgement and blood leakage detection device according to claim 1, wherein the flexible substrate further comprises an extension portion extending outward from the alignment portion and detachably connectable to the conductive terminal, the blood leakage detection electrodes each further comprising a first conduction section formed on the extension portion for being in signal connection with the conductive terminal.

3. The needle dislodgement and blood leakage detection device according to claim 1, wherein the flexible substrate further comprises an extension portion extending outward from the alignment portion, the blood leakage detection electrodes each further comprising a first conduction section formed on the extension portion, the sensor assembly further comprising a signal cable that is detachably connectable to the extension portion to be in signal connection with the first conduction section, the signal cable being detachably connectable to the conductive terminal to set the first conduction section in signal connection with the conductive terminal.

4. The needle dislodgement and blood leakage detection device according to claim 2, wherein the flexible sensor further comprises two body surface detection electrodes that are fixed to a bottom surface of the flexible substrate, the body surface detection electrodes each comprising a second detection section that is formed on a bottom surface of the alignment portion to be electrically engageable with the patient body surface and a second conduction section that is formed on the extension portion to be in signal connection with the conductive terminal, the control unit being in signal connection with the second conduction sections via the conductive terminal and forming, together with the body surface detection electrodes and the patient body surface, a detection circuit and driving the alarm unit to issue an alarm message when one of the body surface detection electrodes detaches from the body surface to open the detection circuit.

5. The needle dislodgement and blood leakage detection device according to claim 4, wherein the blood leakage detection electrodes each comprise a first extension section that is connected between the first detection section and the first conduction section and is formed on a top surface of the extension portion, the body surface detection electrodes each comprising a second extension section connected between the second detection section and the second conduction section and formed on the bottom surface of the extension portion, the flexible sensor further comprising an insulation film that is mounted to the surface of the extension portion and covers the first extension sections and the second extension sections.

6. The needle dislodgement and blood leakage detection device according to claim 4, wherein the control unit comprises an electrical signal detection module that is electrically connected with the conductive terminal and is operable to apply an electrical detection signal to one of the blood leakage detection electrodes, a power interruption protection module that is drivable and enabled to drive the electrical signal detection module to stop outputting the electrical detection signal, a shorting detection module that is electrically connected to the conductive terminal and is operable to output a shorting signal when detecting shorting of the blood leakage detection electrodes, and a control module that is triggerable by the shorting signal to enable the power interruption protection module, the control module being triggerable by the shorting signal to drive the alarm unit to issue the alarm message.

7. The needle dislodgement and blood leakage detection device according to claim 6, wherein the control unit further comprises a detachment detection module that is electrically connected to the conductive terminal and is set in signal connection with the body surface detection electrodes via the conductive terminal, the detachment detection module forming, in combination with the body surface detection electrodes and the patient body surface, the detection circuit and being operable to generate a detachment signal when the detection circuit is opened, the control module being triggerable by the detachment signal to drive the alarm unit to issue the alarm message.

8. The needle dislodgement and blood leakage detection device according to claim 7, wherein the alarm device further comprises a communication unit in signal connection with an electronic device, the control module being triggerable by the shorting signal and the detachment signal to transmit an alarm signal via the communication unit to the electronic device.

9. The needle dislodgement and blood leakage detection device according to claim 8, wherein the communication unit is set in signal connection with the electronic device via cabled communication or wireless communication.

10. The needle dislodgement and blood leakage detection device according to claim 2, wherein the flexible sensor further comprises at least one body surface detection electrode fixed to a bottom surface of the flexible substrate, the body surface detection electrode comprising a second detection section that is formed on a bottom surface of the alignment portion to be electrically engageable with the patient body surface and two second conduction sections that are respectively and electrically connected to two ends of the second detection section and are formed on the extension portion to be in signal connection with the conductive terminal, the body surface detection electrode being connected through the conductive terminal with the control unit to form a detection circuit, the second detection section, when engaging the patient body surface, generating a capacitive coupling effect with respect to the patient so as to cause variation of electrical signal of the detection circuit, whereby when the control unit, upon detecting the variation of the electrical signal of the detection circuit caused by the second detection section detaching from the patient body surface, drives the alarm unit to issue an alarm message.

11. The needle dislodgement and blood leakage detection device according to claim 1, wherein the housing unit comprises housing body to which the conductive terminal, the control unit, and the alarm unit are mounted and a looping member that is mounted to the housing body to loop around the patient.

12. The needle dislodgement and blood leakage detection device according to claim 1, wherein the alignment portion comprises a curved plate that extends annularly or in a curved form to define the needle piercing space that has an opening.

13. The needle dislodgement and blood leakage detection device according to claim 1, wherein the flexible substrate further comprises two flexible wing portions extending outward from the alignment portion to be attached to the patient body surface.

14. The needle dislodgement and blood leakage detection device according to claim 1, wherein the alarm message issued by the alarm unit is alarm lighting or alarm sound.

15. The needle dislodgement and blood leakage detection device according to claim 3, wherein the flexible sensor further comprises two body surface detection electrodes that are fixed to a bottom surface of the flexible substrate, the body surface detection electrodes each comprising a second detection section that is formed on a bottom surface of the alignment portion to be electrically engageable with the patient body surface and a second conduction section that is formed on the extension portion to be in signal connection with the conductive terminal, the control unit being in signal connection with the second conduction sections via the conductive terminal and forming, together with the body surface detection electrodes and the patient body surface, a detection circuit and driving the alarm unit to issue an alarm message when one of the body surface detection electrodes detaches from the body surface to open the detection circuit.

16. The needle dislodgement and blood leakage detection device according to claim 15, wherein the blood leakage detection electrodes each comprise a first extension section that is connected between the first detection section and the first conduction section and is formed on a top surface of the extension portion, the body surface detection electrodes each comprising a second extension section connected between the second detection section and the second conduction section and formed on the bottom surface of the extension portion, the flexible sensor further comprising an insulation film that is mounted to the surface of the extension portion and covers the first extension sections and the second extension sections.

17. The needle dislodgement and blood leakage detection device according to claim 15, wherein the control unit comprises an electrical signal detection module that is electrically connected with the conductive terminal and is operable to apply an electrical detection signal to one of the blood leakage detection electrodes, a power interruption protection module that is drivable and enabled to drive the electrical signal detection module to stop outputting the electrical detection signal, a shorting detection module that is electrically connected to the conductive terminal and is operable to output a shorting signal when detecting shorting of the blood leakage detection electrodes, and a control module that is triggerable by the shorting signal to enable the power interruption protection module, the control module being triggerable by the shorting signal to drive the alarm unit to issue the alarm message.

18. The needle dislodgement and blood leakage detection device according to claim 17, wherein the control unit further comprises a detachment detection module that is electrically connected to the conductive terminal and is set in signal connection with the body surface detection electrodes via the conductive terminal, the detachment detection module forming, in combination with the body surface detection electrodes and the patient body surface, the detection circuit and being operable to generate a detachment signal when the detection circuit is opened, the control module being triggerable by the detachment signal to drive the alarm unit to issue the alarm message.

19. The needle dislodgement and blood leakage detection device according to claim 18, wherein the alarm device further comprises a communication unit in signal connection with an electronic device, the control module being triggerable by the shorting signal and the detachment signal to transmit an alarm signal via the communication unit to the electronic device.

20. The needle dislodgement and blood leakage detection device according to claim 19, wherein the communication unit is set in signal connection with the electronic device via cabled communication or wireless communication.
